(19) United States
(12) Patent Application Publication
(10) Pub. No.: US 2023/0414163 A1
(43) Pub. Date: Dec. 28, 2023

(54) HIGH RESOLUTION MANOMETRY WITH INTRLUMINAL IMPEDANCE (HRMZ) FOR DETERMINING GASTROINTESTINAL TRACT PARAMETERS

(71) Applicant: THE REGENTS OF THE UNIVERSITY OF CALIFORNIA, Oakland, CA (US)

(72) Inventors: Ali ZIFAN, La Jolla, CA (US); Ravinder K. MITTAL, La Jolla, CA (US)

(21) Appl. No.: 18/034,758

(22) PCT Filed: Oct. 1, 2021
(86) PCT No.: PCT/US2021/053106

§ 371 Date: May 1, 2023

Related U.S. Application Data
(60) Provisional application No. 63/107,589, filed on Oct. 30, 2020.

(51) Int. Cl. A61B 5/00; A61B 5/0538; A61B 5/107; A61B 5/03; A61B 5/0536; A61B 5/11

(52) U.S. Cl. CPC A61B 5/4205; A61B 5/0538; A61B 5/4233; A61B 5/7425; A61B 5/037; A61B 5/0536; A61B 5/1107; A61B 5/1076

(57) ABSTRACT

Intraluminal impedance recordings are used to calculate luminal cross-sectional area, or in other words, distension of the esophagus/gastrointestinal tract, during peristalsis using various recording protocols and algorithms derived using the Ohm's law of electricity. Additionally, multiple visual displays of distension-contraction plots of esophageal peristalsis are provided that allows both the relaxation and contraction phase of peristalsis to be easily assessed. These distension-contraction plots can be used to diagnose disorders of the esophagus or other regions of the gastrointestinal tract that result in symptoms such as difficulty swallowing (dysphagia), heartburn, and chest pain, in the case of esophagus. Furthermore, the effects of pharmacological agents/drugs on the distension-contraction measurements can be studied using these protocols and algorithms to treat patients with esophageal symptoms.

### CROSS-REFERENCE TO RELATED APPLICATION

This application claims the benefit of U.S. Provisional Application Ser. No. 63/107,589, filed Oct. 30, 2020, the contents of which are incorporated herein by reference.

### BACKGROUND

The esophagus, an approximately 25-centimeter-long tube, connects the mouth to the stomach. Its major function is to transfer food and other swallowed materials from the mouth and pharynx into the stomach. The upper and lower ends of the esophagus are guarded by upper and lower esophageal sphincter, respectively. The upper esophageal sphincter separates esophagus from pharynx and airway. On the other hand, the lower esophageal sphincters separate the lower end of the esophagus from the stomach. These sphincters are valve like structure and stay always closed except during the act of swallow, belching, regurgitation, and vomiting.

Each act of swallow elicits relaxation of the upper and lower esophageal sphincter, followed by esophageal peristalsis. The latter consists of two phases, an initial inhibition or relaxation phase, which is followed by the contraction phases (ring of closure of esophagus that travels sequentially from the top to the bottom of esophagus). Dysfunction/malfunction of the esophagus leads to difficulty swallowing, chest pain, heartburn, and regurgitation symptoms. Symptoms of heartburn and regurgitation also known as gastroesophageal reflux or GERD are common in the general population. Difficulty swallowing also known as dysphagia is also quite common in the general population.

When a patient with dysphagia symptom goes to the physician for diagnosis; after careful history, physician generally orders various tests to determine the cause of their symptom. Generally, X ray studies, also known as a barium swallow study, is the first test. It assesses the reason for dysphagia such as tumor, strictures, compression of the esophagus from thoracic structures and other possible etiologies that prevent the smooth transfer of swallowed contents into stomach. An upper endoscopy or EGD (esophago-gastro-duodenoscopy) is generally the next test. One can visualize the inside of esophagus and stomach to diagnose various causes of dysphagia and esophageal symptoms. If the barium swallow and upper endoscopy are normal, high-resolution manometry with intraluminal impedance (HRMZ) is the next test ordered. Manometry measures pressures inside the lumen of the esophagus. On the other hand, the impedance part of the HRMZ records transit of the swallowed bolus as it passes along the length of the tube. Prolonged intraluminal impedance recordings of the esophagus are also used to detect GERD, but that is different from the impedance recordings used during HRM recordings to record transit of bolus during swallow-induced peristalsis.

HRMZ is the current gold-standard test to diagnose esophageal motility disorders. These motility disorders include, achalasia esophagus, diffuse esophageal spasm, nutcracker esophagus, esophago-gastric junction outflow obstruction (EGJOO) and ineffective esophageal motility disorders. Studies shows that large number of patients with dysphagia have normal esophageal function testing, that include barium swallow, EGD examination and HRMZ recordings. Our estimate is that more than 50% of patients referred for dysphagia testing have normal recordings, and these patients are thought to have functional dysphagia, which implies dysphagia of unknown origin.

The initial or the first phase of esophageal peristalsis, i.e., the relaxation phase of peristalsis allows opening of the esophagus to accommodate/intake the bolus and is not accurately measured by the HRMZ recordings. The current limitation of HRMZ recordings in clinical use is that it accurately assess only the contraction but not the relaxation phase of peristalsis. The relaxation of esophagus allows it to distend with minimal resistance so that the bolus can pass through the esophagus.

### SUMMARY

Systems and methods are described herein that allow one to visually display and quantify distension contraction parameters in the diagnosis of dysphagia of unknown origin. Studies have shown that the degree of esophageal distension ahead of contraction is a surrogate of relaxation and can be measured from the intraluminal esophageal impedance part of HRMZ recordings. A methodology is described herein to measure distension of the esophagus during peristalsis using intraluminal impedance measurements. Using this methodology, the characteristics of swallow-induced distension-contraction profiles have been described in normal healthy subjects, e.g., the amplitude and duration of distension increases from proximal to distal esophagus. Furthermore, it has been found that there is a unique temporal relationship between distension and contraction, i.e., a wave of distension travels in close relationship to contraction, especially in the Trendelenburg position (head end of the subjects lower than the foot end). Computer software has been developed that can generate distension-contraction profiles of the esophagus during swallow-induced peristalsis, quantify the amplitude of distension, and the temporal relationship between distension-contraction waveforms from the HRMZ studies. Studies show that many patients with difficulty swallowing and who have normal barium swallow, upper endoscopy and HRMZ recordings (performed according to the current protocol), have abnormalities in the relaxation phase of peristalsis. The esophagus does not distend as well in these patients as it does in normal healthy subjects.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter. Furthermore, the claimed subject matter is not limited to implementations that solve any or all disadvantages noted in any part of this disclosure. It will be appreciated that the above-described subject matter may be implemented as a computer-controlled apparatus, a computer process, a computing system, or as an article of manufacture such as one or more computer-readable storage media. These and various other features will be apparent from a reading of the following detailed description and a review of the associated drawings.

### BRIEF DESCRIPTION OF THE DRAWINGS

FIG. 1 schematically illustrates an HRMZ catheter situated in an esophageal lumen to illustrate the principle of intraluminal impedance measurements.

FIG. 2 shows the parameters related to the resistance of a cylindrical medium.

FIG. 3A shows a mesh model of a catheter inside the esophagus alongside a bolus at the lower esophagus; FIG. 3B shows the electrodes of the catheter; FIG. 3C shows forward, and inverse models and FIG. 3D shows the reconstructed conductivity image showing the bolus.

FIG. 4 shows an example of an impedance pressure heatmap, with the simultaneous use of two non-overlapping colormaps (depicted in pseudo-color).

FIG. 5 shows an example of a plot of impedance gradient streamlines overlaid on pressure (depicted in pseudo-color).

FIG. 6 shows an example of a distension-contraction plot of a 10 cc saline swallow (depicted in pseudo-color).

FIG. 7 shows an example of a distension-contraction plot with distension depicted as a waveform and pressure as a heatmap.

FIGS. 8 and 9 shows examples of distension-contraction montages of a 10 cc saline swallow.

FIG. 10 shows an example of a distensibility plot of a 10 cc saline swallow.

FIG. 11 shows an example of a distension-tension plot of a 10 cc saline swallow with distension shown as a waveform and tension as heatmap (depicted in pseudo-color).

FIG. 12 shows a plot of illustrative esophageal length tension loops.

FIG. 13 shows a plot of illustrative esophageal pressure-radius lofts.

### DETAILED DESCRIPTION

### Introduction

There are several reasons why visualizing intraluminal distension is clinically important in assessing GI motility disorders:
1. Swallowing and distention of the esophagus induced LES relaxation: proper lower esophageal sphincter (LES) relaxation is necessary in order to allow the passage of food or liquid into the stomach. However, the LES muscle does not always work perfectly. Sometimes it is too weak to stay completely closed, allowing reflux of gastric contents into the esophagus to occur. It is also known that esophageal distention at the level of either striated or smooth muscle segments can elicit LES relaxation. Thus, poor distension of the esophageal wall can be the cause of difficulty with bolus transport in the esophagus and hence can cause difficulty swallowing or dysphagia. Therefore, being able to visualize bolus transport and quantify regional distension in the esophageal lumen would be an invaluable tool in order assess esophageal motility problems in clinical practice.
2. Cross sectional area (CSA) of the esophagus or distension of the esophagus ahead of the contraction is an indirect measure of the relaxation phase of peristalsis, which is not measured by the current recording techniques. Distension of the esophagus is equivalent to the size of the highway through which cargo (bolus) must travel to reach its destination, i.e., stomach. A poorly distending esophagus is similar to a narrow highway through which bolus must be squeezed through by the oncoming peristaltic contraction to reach the stomach. Therefore, abnormalities in the distension phase of peristalsis would result in difficulty of passage of food and other swallowed material from the mouth into the stomach. Therefore, accurate measurement of the distension phase of peristalsis is of paramount importance and is not accurately measured by the currently available techniques.
3. Movement of bolus head and tail: while at rest, the esophageal body has a small amount of tone, it is mostly quiescent, may contain small amounts of air and reflect intrathoracic pleural pressures. X ray fluoroscopic examination shows that drinking a bolus of liquid or barium in the upright position, the bolus travels quickly from the pharynx to the esophagus, and then into the stomach. The radiologic examination reveals that the head of a liquid barium bolus normally enters the distal esophagus within a few seconds of initiating the swallow, which is mostly due to the powerful “pump like” function of the pharynx and aided by gravity. A few seconds later, sequential contraction of the esophagus (peristalsis) sweeps down the length of the esophagus and propels the bolus into the stomach, bolus of solid food also requires peristaltic contraction for its propulsion into the stomach. It takes approximately 8 to 10 seconds from the initiation of swallow for the bolus to enter into the stomach. The head of the liquid barium bolus moves much faster than its tail in the upright posture, but the two move at about the same speed in the recumbent posture and more so in the Trendelenburg position. Thus, visualizing the movement of the head end of bolus and tail of the bolus are also relevant in assessing disorders of esophageal peristalsis. Our studies show that in patients with dysphagia of unknown origin the esophagus does not distend well (narrow esophagus) and hence bolus can travel with a higher bolus flow rate and velocity, resulting in earlier arrival of the head end of bolus in the distal esophagus. In some patients the bolus gets stuck in the distal esophagus due to a closed lumen of the esophagus.
4. Velocity of bolus flow and biomechanical properties of the esophageal wall: poor distension results in a narrow esophagus, which alters the bolus flow characteristics, 1) bolus flows rapidly through a narrow esophagus which results in faster arrival of the bolus in the distal esophagus, and 2) reduced distension of the esophagus and a higher pressure in the lumen of the esophagus suggests poor distensibility of the esophageal wall and greater esophageal wall tension during transport. These changes may result in a sensation of dysphagia or obstructed bolus and possibly esophageal pain.

Procedure

The methods described herein may be performed on a subject in the following manner. After placement of the HRMZ catheter via the nose into the esophagus and stomach of the subjects, the subject is asked to swallow saline of known concentration (e.g., 0.5 N saline and 0.1 N saline). One can use various volumes of a swallowed bolus, e.g., 5 ml, 10 ml and 15 ml saline. Instead of a saline bolus, one can use a viscous bolus of e.g., 0.5 N saline conductivity to assess the cross-sectional area of the esophagus and bolus flow characteristics.

A typical HRMZ catheter generally has 36 pressure sensors, located 1 cm apart, and 18 impedance electrodes (2 cm apart). More generally, however, HRMZ catheters may be employed that have any number of pressure and impedance sensors. The subject may be positioned in the supine or Trendelenburg position during these recordings. The latter position is advantageous when studying a saline bolus because the air and saline are separated as they traverse through the esophagus, which increases the accuracy of the cross-sectional area (CSA) measurements from the recorded impedance values. One can also use swallows with two concentrations of saline (heated to body temperature in a water bath), e.g., 0.1 N and 0.5 N, of varying bolus volumes (e.g., 5 cc, 10 cc and 15 cc), with the subject lying down in the Trendelenburg position to improve the accuracy of the CSA measurement obtained from the recorded impedance values. The CSA of the esophagus at each electrode pair is estimated by solving two algebraic Ohm's law equations, resulting from the two saline solutions. The CSA estimate can be improved by using a correction factor calculated from the in vitro (using the same methodology) testing in glass test tubes of a known CSA.

### CSA Estimation in the Esophagus

Multichannel intraluminal impedance (MII) is the current gold standard for assessing bolus transit/clearance and monitoring acidic/non-acidic reflux monitoring in the esophagus. However, MII in the currently used format can neither resolve bolus shape nor luminal distention of the esophagus. Multi-channel intraluminal impedance (MII) was introduced to the GI community in the early 1990s to resolve previous limitations of esophageal function tests, such as the lack of ability to detect bolus transit and characteristics of the refluxate (liquid, gas, or mixed) and nonacidic GER. MII along with manometry allows determining the presence of a bolus and its relationship with peristalsis. MII detects changes in conductivity provoked by bolus presence in the esophageal lumen. Traditional intraluminal impedance measurements use ring electrodes separated by 2 cm. These ring electrodes can have different diameters (ranging generally from 2 to 4 mm mm) and various heights (e.g., 4 mm). A typical MII catheter consists of 8 stainless steel rings longitudinally located at 2 cm intervals. More complex mathematical models of MII probes have also been developed, as discussed for example, in Kassab G. S., Lontis E. R., Gregersen H. 2004, “Measurement Of Coronary Lumen Area Using An Impedance Catheter: Finite Element Model And In Vitro Validation,” Ann. Biomed. Eng. 32, 1642-1653. These models demonstrate the impact on the measurements caused by electrode spacing, electrode length, number of channels and the radius of the catheter. MII along with manometry (in the form of a combined HRMZ system) allows the presence of a bolus (not its shape) to be determined and its relationship with peristalsis.

MII detects changes in conductivity caused by the bolus presence in the esophageal lumen. In the absence of a bolus the impedance is determined by the esophageal lining and intra-thoracic structures. The presence of bolus decreases impedance due to its high ionic content. MII measurements employ alternating current applied between two ring metal electrodes arranged longitudinally on a probe. The following physical (electrical) principles may be used to calculate luminal cross-sectional area/distension during bolus transport in the lumen.

Electric flux (Φ) can be defined as:

Φ=EA cos θ (1)

where θ is the angle formed by the normal to the surface cross-sectional area A and the electric field E.

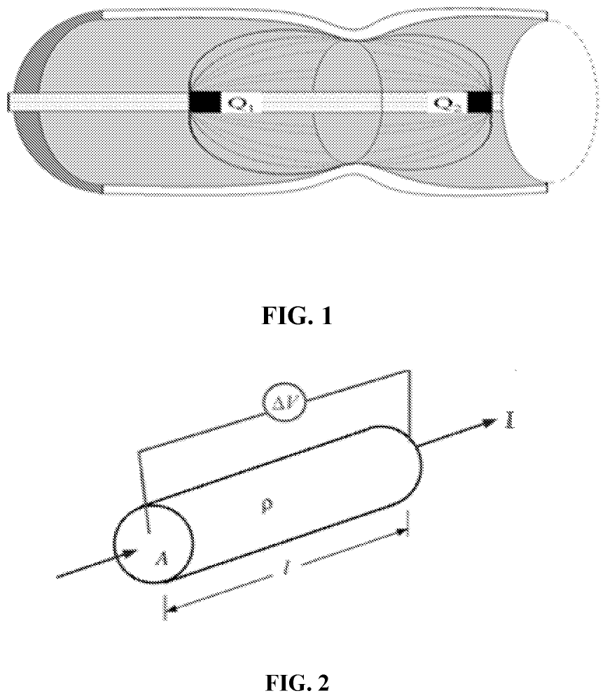

As illustrated with reference to FIG. 1, the impedance between the electrodes depends on the bolus composition, and changes in the cross-sectional area of the esophageal lumen during peristalsis and bolus transit. The measuring current utilized by MII in a HRMZ system generally has an amplitude of 6 μA and a frequency ranging from 1 kHz to 2 kHz. The impedance between the two longitudinally arranged ring electrodes is then calculated as:

$$Z = \frac{U}{I} = f ( {Q}_{x} ) \qquad ( 2 )$$

where Z is impedance, U represents the electric potential, I is electric current and Qₓ is the cross-sectional area of the esophageal lumen. However, finding the function ‘ƒ’ is not a trivial task. The goal is to find the function (a regression) that relates esophageal cross-sectional distention and impedance measurements, as illustrated in FIG. 2 for a cylindrical medium.

When an electric current passes through the length of the esophagus, it experiences an opposition or impedance (Z) to its flow, which results in the loss of energy. This impedance is not only due to the segment of the esophagus lying in between the electrode pairs, but also the tissue/organs in proximity of the electric field, because of the leakage of current into the surrounding body. In general, the impedance will be complex, comprised of two components: Z=R+jX, resistive (energy dissipating) and reactive (energy conserving) parts, where the magnitude and phase of the response will often be frequency dependent. At low frequency like those used in common HRMZ systems, current passes through the extracellular fluid (ECF) space, and does not penetrate the cell membrane, diminishing their capacitive effects (X_{C}=1/iωC≈0, ω being the angular frequency and capacitance denoted by C). Thus, impedance becomes equivalent to resistance. Similarly, the inverse of impedance (i.e., admittance) becomes equivalent to the inverse of resistance, namely conductance denoted by G. Moreover, as stated above, the resistance of a geometrical system is related to the conductor length, its cross-sectional area, and its intrinsic properties, namely resistivity,

R=L×ρ/CSA (3)

where ρ denotes the resistivity (Ω-m) of the conductor material, L the length of the conductor (m), and CSA is the cross-sectional area (m²). Therefore, one can use Eq. (3) to calculate CSA provided all the other parameters in the equation are known.

Esophageal electrical impedance (or equivalently resistance) can be obtained from MII measurements using HRMZ systems. However, based on the previous discussion, the total resistance will be a weighted sum of all the tissue/organs falling in the electric field between the electrode pair, rather than solely the esophagus, causing inter-patient impedance value variability, especially baseline differences.

As explained below, in some embodiments, the systems and methods described herein may employ a procedure in which measurements are made while a single bolus is swallowed while in other embodiments the systems and methods described herein may employ a procedure in which measurements are made while two boluses are swallowed sequentially. Each of these embodiments will be discussed in turn.

### CSA Estimation Using a Single Saline Concentration Bolus

Two embodiments are described herein in which a single bolus is swallowed. In the first case discussed below, the CSA is determined by taking into account the conductance of the perimeter tissues and organs surrounding the esophagus and in the second embodiment the CSA is determined by ignoring the conductance of the tissues and organs surrounding the esophagus.

Assuming the esophagus has a conductance (inverse of resistance) denoted by Gₑₛₒ and the surrounding tissue and organs has a conductance denoted by Gₚₑᵣᵢₘ, and the measured conductance as Gₘₑₐₛ,

Gₘₑₐₛ=Gₑₛₒ+Gₚₑᵣᵢₘ (4)

At time t₀, at baseline, assuming the absence of any bolus within the esophageal lumen and a collapsed lumen (CSA=0), based on equation (3), Gₑₛₒ becomes,

Gₜ_{₀}^{eso}=CSA/L×ρ=0 (5)

Substituting (5) to equation (4),

Gₜ_{₀}^{meas}=Gₜ_{₀}^{perim} (6)

Next, at time t₁, during a bolus swallow (e.g., 0.5 N), of resistivity ρ_{0.5 N saline},

$${G}_{{t}_{1}}^{m e a s} = {G}_{{t}_{1}}^{e s o} + {G}_{{t}_{1}}^{p e r i m} \to {G}_{{t}_{1}}^{m e a s} = {[ ( \frac{C S {A}_{e s o}}{L {ρ}_{0.5 N}} ) ]}_{{t}_{1}} + {G}_{{t}_{1}}^{p e r i m} \qquad ( 7 )$$

Solving equations (6) and (7), and assuming the surrounding tissue conductivity stays the same (Gₜ_{₁}^{perim}=Gₜ_{₀}^{perim}), we obtain the esophageal luminal CSA,

$$\to C S {A}_{e s o} = L \frac{( {G}_{{t}_{1}}^{m e a s} - {G}_{{t}_{0}}^{m e a s} )}{{σ}_{0.5 N}} \qquad ( 8 )$$

Where CSAₑₛₒ denotes the CSA of the esophagus at a particular height, between an electrode pair (L distance between them), and σₛₐₗᵢₙₑ denotes the conductivity (inverse of resistivity) of the saline solutions used.

The value of CSAₑₛₒ obtained using Equation (8) maybe refined to improve its accuracy using a correction factor that is obtained by carrying out the same process in vitro in glass tubes of known diameter. In this way the CSA estimation error is calculated for each tube (based on the electrode spacing, shape, etc.). Next, non-linear regression is carried out to obtain the correction factor for each tube and CSAs in-between. Finally, in in-vivo, the use of Equation (8) combined with the correction factor estimated in-vitro, produces the final CSA at any electrode pair site.

A calculation of the CSA that ignores the perimeter tissues and organs around the esophagus will now be described. Assuming the esophagus has a conductance (inverse of resistance) denoted by Gₑₛₒ and the surrounding tissue and organs has a conductance denoted by Gₚₑᵣᵢₘ=0, based on equation (4), the measured conductance Gₘₑₐₛ, becomes,

Gₘₑₐₛ=Gₑₛₒ (9)

At time t₀, substituting (9) into (3),

Gₜ_{₀}^{eso}=CSAₑₛₒ/L×ρ=Gₜ_{₀}^{meas} (10)

So, CSA becomes,

$$C S {A}_{e s o} = L \frac{( {G}_{{t}_{0}}^{m e a s} )}{{σ}_{0.5 N}} \qquad ( 11 )$$

As before, the value of CSAₑₛₒ obtained using Equation (11) may be refined to improve its accuracy using a correction factor that is obtained by carrying out the same process, i.e., in vitro in glass tubes of known diameter, and the CSA estimations error using Eq (11) is calculated for each tube. Next, non-linear regression is carried out to obtain the correction factor for each tube and CSAs in-between. Finally, in in-vivo, the use of Equation (11) combined with the correction factor estimated in-vitro, produces the final CSA at any electrode pair site.

Next, embodiments of the systems and methods described herein are presented in which measurements are made while two boluses are swallowed sequentially. These embodiments employ a modified technique originally introduced by Kassab et al (referenced above) in cardiology to measure the CSA of coronary vessels using a specialized catheter. This technique is refined and adapted to measure the CSA of the esophagus during peristalsis using HRMZ measurements. The technique introduced by Kassab et al. for coronary arteries (see Kassab G. S., Lontis E. R., Horlyck A., Gregersen H. 2005, “Novel Method For Measurement Of Medium Size Arterial Lumen Area With An Impedance Catheter: In Vivo Validation,” Am. J. Physiol. Heart Circ. Physiol. 288, H2014-2020, uses two bolus injections of saline solutions with known electrical conductivities to transiently displace blood and to effectively minimize the hemodynamics-induced blood conductance alterations for analytical determination of vessel cross-sectional area (CSA) and the electric current leakage through the vessel wall and surrounding tissue (parallel conductance).

In accordance with this procedure, at time t₁, using e.g., a 0.1 N volume saline of known resistivity ρ_{0.1 N saline}, using Eq. (4) we obtain the following:

$${G}_{{t}_{1}}^{m e a s} = {G}_{{t}_{1}}^{e s o} + {G}_{{t}_{1}}^{perim} = {[ ( \frac{C S {A}_{e s o}}{L {ρ}_{0.1 N}} ) ]}_{{t}_{1}} + {G}_{{t}_{1}}^{perim} \qquad ( 12 )$$

Next, doing the same for time t₂ using the same formulation, inserting the same volume with a different concentration (e.g., 0.5 N) of resistivity ρ_{0.5 N saline},

$${G}_{{t}_{2}}^{m e a s} = {G}_{{t}_{2}}^{e s o} + {G}_{{t}_{2}}^{perim} \to {G}_{{t}_{2}}^{m e a s} = {[ ( \frac{C S {A}_{e s o}}{L {ρ}_{0.5 N}} ) ]}_{{t}_{2}} + {G}_{{t}_{2}}^{perim} \qquad ( 13 )$$

Solving equations (12) and (13), and assuming the surrounding tissue conductivity stays the same (Gₜ_{₁}^{perim}=Gₜ_{₂}^{perim}=K), we obtain the esophageal luminal CSA,

$$\to C S {A}_{e s o} = L \frac{( {G}_{{t}_{2}}^{m e a s} - {G}_{{t}_{1}}^{m e a s} )}{( {σ}_{0.5 N} - {σ}_{0.1 N} )} \qquad ( 14 )$$

With CSAₑₛₒ denoting the CSA of the esophagus at a particular height, between an electrode pair (where L is distance between them), and σₛₐₗᵢₙₑ denoting the conductivity (inverse of resistivity) of the saline solutions used.

Once again, the value of CSAₑₛₒ obtained using Equation (14) may be refined to improve its accuracy using a correction factor that is obtained by carrying out the same process in vitro in glass tubes on known diameter, and the CSA estimations error using Eq (14) is calculated for each tube. Next, non-linear regression is carried out to obtain the correction factor for each tube and CSAs in-between. Finally, in in-vivo, the use of equation (14) combined with the correction factor estimated in-vitro, produces the final CSA at any electrode pair site.

To expand the capability to the entire duration of the swallow, there is one important hurdle to overcome, and that is the ‘duration’ of the swallow for the two saline boluses, though similar, may not be exactly the same. The latter means that prior to subtraction, the corresponding waveforms must be temporally aligned for all the impedance channels. In one implementation, “dynamic time warping” described in Myers C S, Rabiner L R, “A Comparative Study Of Several Dynamic Time-Warping Algorithms For Connected Word Recognition, The Bell System Technical Journal 1981; 60:10.), may be employed for this purpose, which is a well-known technique in speech processing to find an optimal alignment between two waveforms. In the present case dynamic time warping may be used to align the two saline solution waveforms, after which the CSA estimation process can be performed using Eq. (14). Once the two-bolus protocol is carried out during routine esophageal HRMZ testing, a computer program can be used to present a display of the bolus as it transverses the length of the esophagus. In this way the previous CSA estimations will be more robust, provided the subject is lying down in the Trendelenburg position, as it allows separation of swallowed air from the saline bolus. Note that as the viscosity of the swallowed bolus increases, recordings can be carried out in the supine position because a viscous bolus in the supine position travels the esophagus in a manner similar to a saline bolus in the Trendelenburg position.

### CSA Estimation Using Esophageal Impedance Tomography

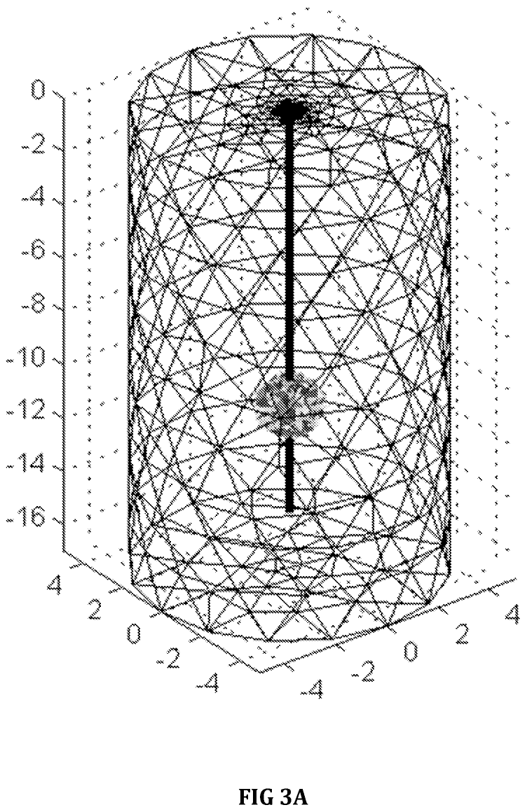

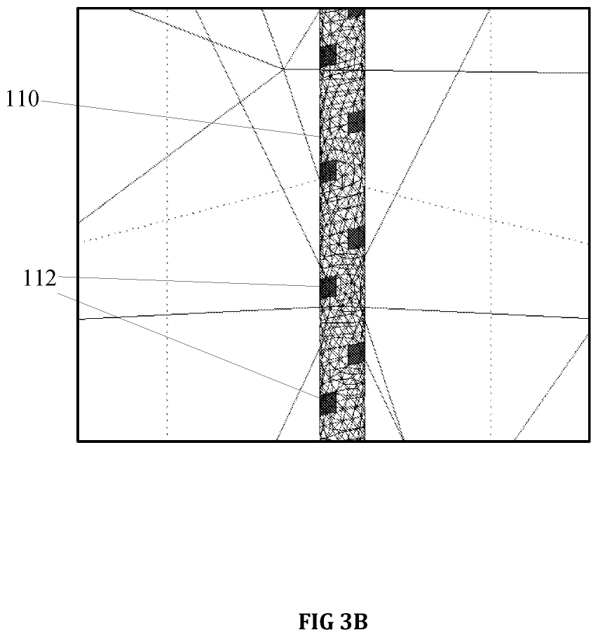

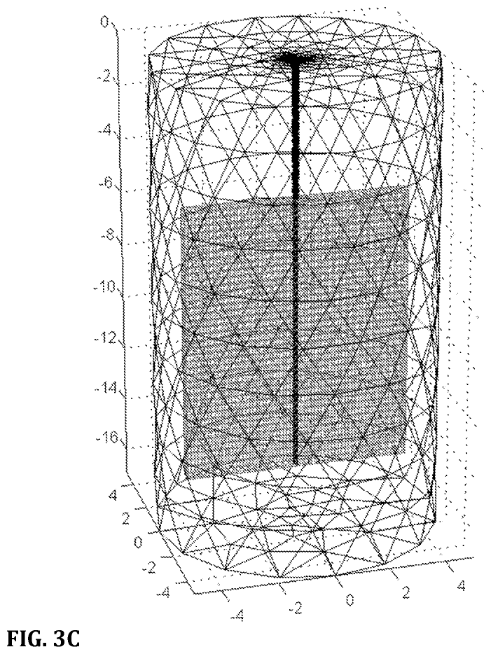

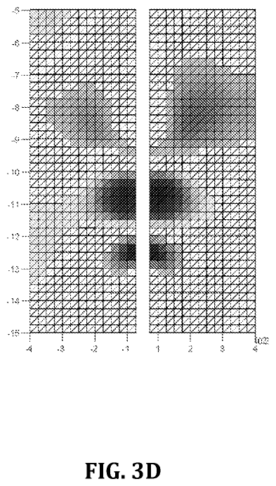

A more advanced method of calculating CSA takes advantage of the conductivity changes within the esophageal lumen with the swallowing of a liquid or solid bolus. This method uses inverse modeling techniques employed in soft-field imaging. This formulation leads to the reconstruction of conductivity (change) images, where the bolus can be subsequently segmented out using computer vision techniques. The latter can be achieved using the same catheter currently used, inserted nasally, with a different current-injection voltage-pickup protocol. In particular, catheters currently used in HRMZ have a single circular band of electrodes. For use in esophageal impedance tomography, however, each ring of electrodes will be composed of multiple electrodes in each of the ring. Such an arrangement is shown in FIG. 3B, which shows the catheter 110 and the electrodes 112.

At each esophageal level, current is injected into one electrode pair and the voltages between other electrodes are recorded. For an adjacent protocol for example, the injection can be successively shifted so that all electrode pairs are used using a single frequency (50 kHz) or multi-frequency (up to 1 MHz). The governing equation for the voltage field produced across the esophageal body Ω is:

∇(σ−ωε)∇ϕ=0 (15)

where σ is the electric conductivity of the medium, ϕ is the electric potential, ω is the frequency, and ε is the electric permittivity. To estimate σ (i.e., esophageal tissue conductivity), the following two problems must be solved: forward and inverse. The forward problem is the problem of determination of voltage distribution for a known conductivity distribution in the oesophagus, while the inverse problem consists of conductivity image reconstruction using the measured voltages at the surface of the catheter.

If we represent the forward operator g by g(m)=d, where m is the model and d is the boundary measurement voltage vector, the goal is to come up with a model, which yielded the actual measured voltages, denoted by d_{T}, the simplest approach is to minimize the following sum, which is the minimum of the sum of square errors,

∥d_{T}−g(m)∥_{F}² (16)

where F denotes the Frobenius norm.

Now, assuming no model null-space and that only the data misfit term as described in equation (7) is required to solve the inverse problem, if we could somehow linearize the g operator we could use linear methods such as the conjugate gradient (CG) to derive the critical points of Eq. (16). This can be done by linearizing the forward problem in the neighbourhood of a reference model m₀, using a Taylor expansion,

$${{( m ) = g ( {m}_{0} ) + \frac{\partial g}{\partial m} \vert}_{m = {m}_{0}} ( m - {m}_{0} ) + \frac{{\partial }^{2} g}{\partial {m}^{2}} \vert}_{m = {m}_{0}} {( m - {m}_{0} )}^{2} + … \\ \approx g ( {m}_{0} ) + ∇ g ( {m}_{0} ) ( m - {m}_{0} ) + … \qquad ( 17 )$$

Ignoring higher order terms,

g(m)=g(m₀)+G(m−m₀) (18)

where G is a rectangular matrix that gives the sensitivity of the forward problem to the model parameters at m=m₀:

$${{G}_{ij} = \frac{\partial {g}_{j}}{\partial {m}_{i}} \vert}_{m = {m}_{0}} \qquad ( 19 )$$

Next, we can use this Taylor expansion to linearize the inverse problem,

d=g(m)≈g(m₀)+G(m−m₀) (20)

Let, δd=d−g(m₀); δm=m−m₀ denote the perturbation, so

δd=Gδm (21)

which is the linearized inverse problem for the perturbation of m about m=m₀.

As the problem is ill-posed (small errors in the measurements may introduce large errors in the reconstruction) the minimization of the voltage error in equation (17), is not likely to yield any good results. This is because in practice linear least squares calculations usually involve singular matrices or matrices that are numerically singular (small eigenvalues). For a unique solution, we must add some additional information regarding the conductivity which is independent of the data known as the prior. Regularization mitigates these singularities. This can be done by discarding small eigenvalues or one can penalize the size of the solution as well as the data misfit. In other words, the minimization problem of (17) can be written as:

min(∥δd_{T}−G(δm)∥²+λ∥Rδm∥²) (22)

The first term of (22), is the data misfit, and the second term is referred to as the regularization term. The fudge factor (or hyperparameter) λ controls the tradeoff between the two terms and not only considers the possibility of minimizing the norm itself, but the norm of some linear function (i.e., R) of the mode. If R≡∂ⁿ, n=0, 1, 2, . . . and ∂ⁿ is an n-th order discrete difference operator. In this case, the second term in equation (22) penalizes the slope, roughness, or higher order derivatives of the model. This would be useful if one seeks a smooth solution. Also, sensitivity analysis may be carried out to assess and find the optimal configuration of electrode shape, and different current injection, voltage pick up protocols using an esophageal phantom.

A finite element simulation results of the previously discussed approach is shown in FIGS. 3A-D, where the bolus is represented by a circular inclusion located at the depth of −11 cm with a radius of 1.5 cm, and subsequently adding 12 dB Gaussian pseudorandom noise with varying seeds. To obtain the true resistivities (or conductivities), both the forward and inverse models should be solved. Furthermore, with the careful selection of edge-preserving priors, allows more coherent and contrast regions of bolus presence.

As it is clear from FIGS. 3C-D, the reconstructed conductivity correctly localized the bolus, as well as its shape to a good degree. The benefit of the above technique is that it can be expanded to multi-frequency to allows for the characterization of not only the bolus, but the esophageal wall tissue (e.g., changes in perfusion, which also cause conductivity changes) and the surroundings in real time, allowing the visualization of both liquid and solid boluses.

It should be noted that when esophageal impedance tomography is employed using a solid bolus, it is not necessary or advantageous to have the patient lying down in the Trendelenburg position or necessarily take a bolus of known conductivity.

Determination of Esophageal Parameters

Several parameters associated with the esophagus can be determined from the measurements obtained using the systems and techniques described herein. For instance, LaPlace's law can be used to calculate the tension in the walls of the esophagus. This geometrical law, applied to a tube or pipe, states that for a given internal fluid pressure, the wall tension will be proportional to the radius of the vessel. So, after the calculation of the cross-sectional area (assuming a circular geometry), the radius of the esophageal wall at each location can be estimated and multiplied by the pressure at the same sensor location. This can be carried out with or without the subtraction of pressure values from that of a reference esophageal pressure point at each sensor location, prior to the swallow (pharyngeal opening).

Another parameter that may be determined is distensibility in the esophagus. Once the cross-sectional area of the esophageal wall is obtained at each location, distensibility can be obtained by dividing the CSA with pressure (CSA/pressure). This can be carried out with or without the subtraction of pressure values from that of a reference esophageal pressure point at each sensor location, prior to the swallow (pharyngeal opening).

The luminal cross-sectional area (length) and tension at each of any number (e.g., 36) of locations in the esophagus can be determined and displayed. The tension is calculated as luminal radius (derived from the cross-sectional area) times pressure. These length tension loops are reflective of work done by the esophageal muscle at each location in the esophagus. Likewise, the radius (length) and pressure at each of any number (e.g., 36) of locations in the esophagus can be determined and displayed. The area of these loops is reflective of work done by the esophageal muscle at each location in the esophagus, or their sum at a particular region of the esophagus. Additionally, the luminal radius (length) and distensibility at each of any number (e.g., 36) of locations in the esophagus can also be determined and displayed as length distensibility loops.

The various esophageal extracted parameters (e.g., pressure or pressure-derived parameters, impedance or impedance derived parameters, voltage, current, etc) may be imported, visualized (displayed) and analyzed on any suitable and convenient computer processing device, including, without limitation, personal computers, tablets, smartphones, smart glasses and other hand-held or wearable devices.

### Visualization of Distension and Contraction Phases of Peristalsis

The HRMZ recordings and measurements obtained using the systems and techniques described herein can be analyzed to generate plots of distension-contraction parameters that can be displayed in a variety of different ways. These plots can be generated by software that can be executed on any suitable and convenient computer processing device, which, as previously noted, may include, without limitation, personal computers, tablets, smartphones, smart glasses and other hand-held or wearable devices. Among other things, the software program can be used to generate distension-contraction profiles of the esophagus during peristalsis, quantify the amplitude of distension, and the temporal relationship between distension-contraction waveforms. A number of illustrative plots that can be generated and displayed will be described below.

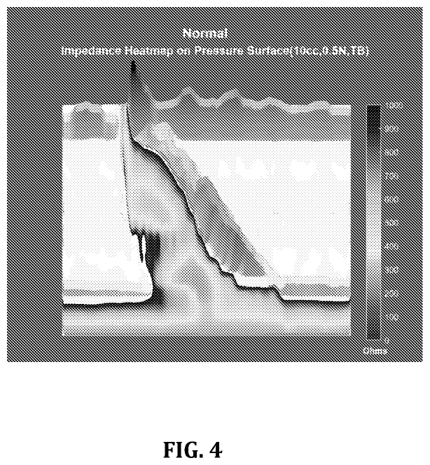

FIG. 4 shows an illustrative display of an impedance pressure heatmap for the simultaneous visualization of impedance and pressure, depicted using non-overlapping colormaps (and illustrated in FIG. 4 using pseudo-color). In contrast, conventional displays present pressure as a heatmap and impedance as a single shade of a specific color (e.g., purple)). The display shown in FIG. 4 can be visualized as an image in 2D or a surface in 3D.

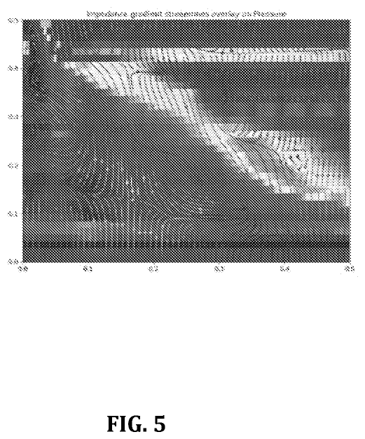

FIG. 5 shows an illustrative display of impedance gradient streamlines overlaid on a pressure heatmap (depicted in FIG. 5 using pseudo-color). The streamlines allows the quick visualization of a bolus moving through regions of low resistance that allow more flow by moving along the direction of the impedance gradient field. This is accomplished by using streamlines of the gradient field achieved using the forward Euler prediction. This also allows the use of topographical streamline analysis methods to extract further information and features from the curves.

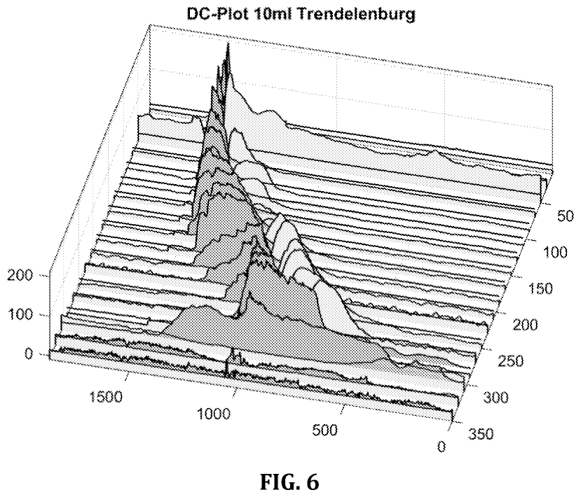

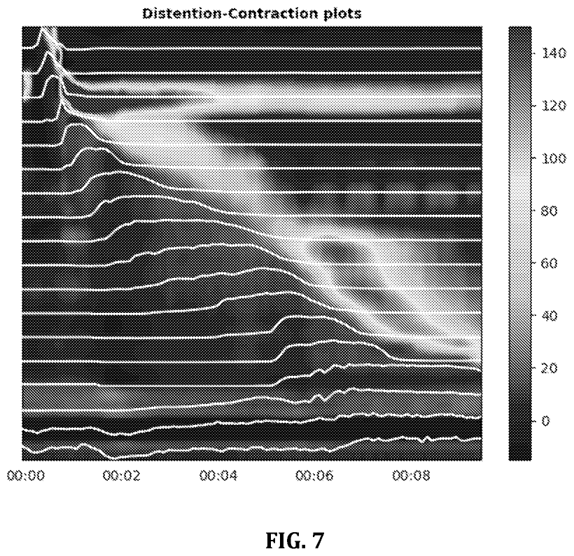

In yet another example 2D and 3D distension-contraction plots can be generated and displayed. The simultaneous visualization of both esophageal distension and contraction during peristalsis can be accomplished by displaying both contraction and distension as signals/waveforms, or distension as a waveform and pressure as a heatmap, or distention as heatmap and pressure as a waveform. FIG. 6 shows an illustrative distension-contraction plot of a 10 cc saline swallow, with distension in one color and contraction in another color (depicted in FIG. 6 using pseudo-color). Likewise, FIG. 7 shows an illustrative distension-contraction plot with distension as a waveform and pressure as a heatmap (depicted in FIG. 7 using pseudo-color).

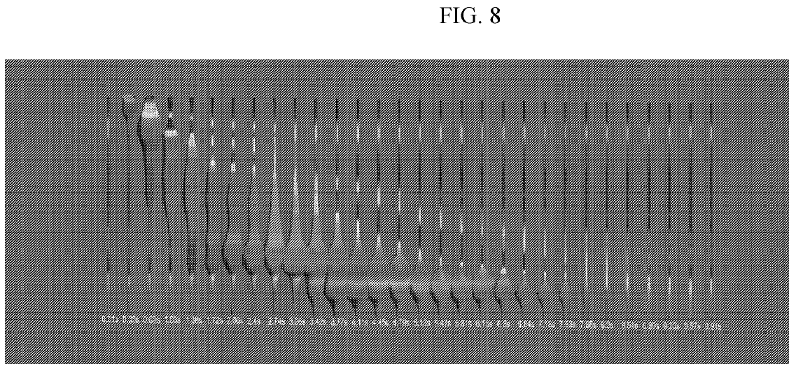

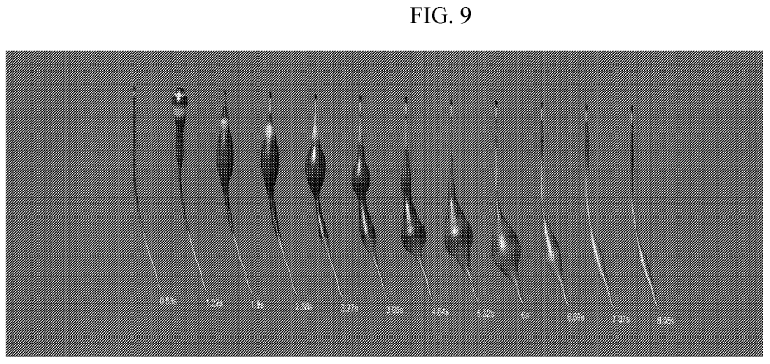

FIGS. 8 and 9 show illustrative distension-contraction montages (both cylindrical and realistic geometry) of a 10 cc saline swallow. FIG. 8 shows a normal subject and FIG. 9 depicts a patient suffering from nutcracker esophagus. These montages can be depicted in 2D, 3D or as a video. The montage can visualize an entire swallow cycle at specified time intervals. In this form of visualization, distension of the esophagus can be displayed in either a cylindrical (mesh) geometry or a realistic anatomical esophageal geometry. Simultaneously, the pressure at each sensor location can be mapped on the mesh for the simultaneous visualization of distension-contraction in another format.

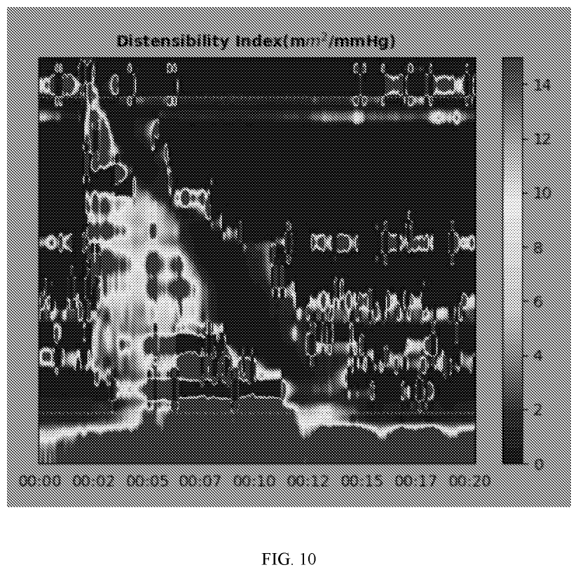

Yet another feature that may be visualized is distensibility during an entire swallow, which may be presented either as an image or at each sensor location to be mapped to the esophageal distension mesh as previously described, but with the overlay of distensibility on the mesh instead of pressure. This feature can be displayed as a single image showing the whole swallow, or as a video with a specified frame rate. FIG. 10 is an illustrative distensibility plot of a 10 cc saline swallow.

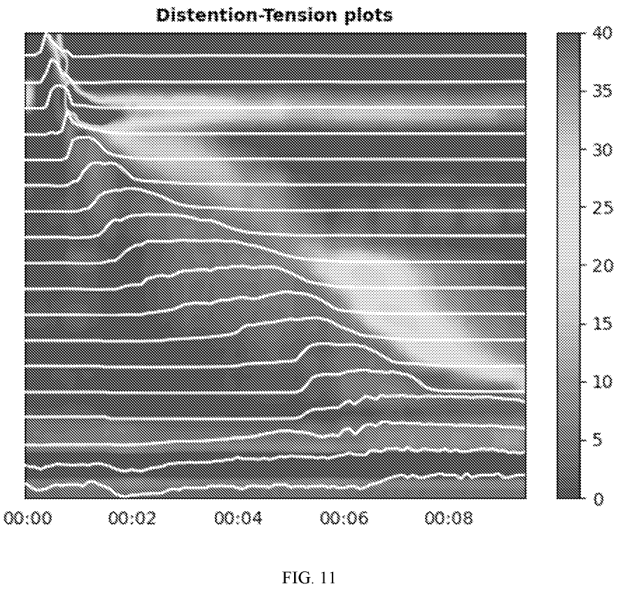

Another feature that may be visualized is the tension during an entire swallow, which may be presented either as an image or at each sensor location to be mapped to the esophageal distension mesh as previously described, but with the overlay of distensibility on the mesh instead of pressure. This feature can be displayed as a single image showing the whole swallow or as a video with a specified frame rate. Distension can also be overlayed on a tension heatmap as shown in FIG. 11, which is an illustrative distension-tension plot of a 10 cc saline swallow where distension is shown as a waveform and tension as a heatmap.

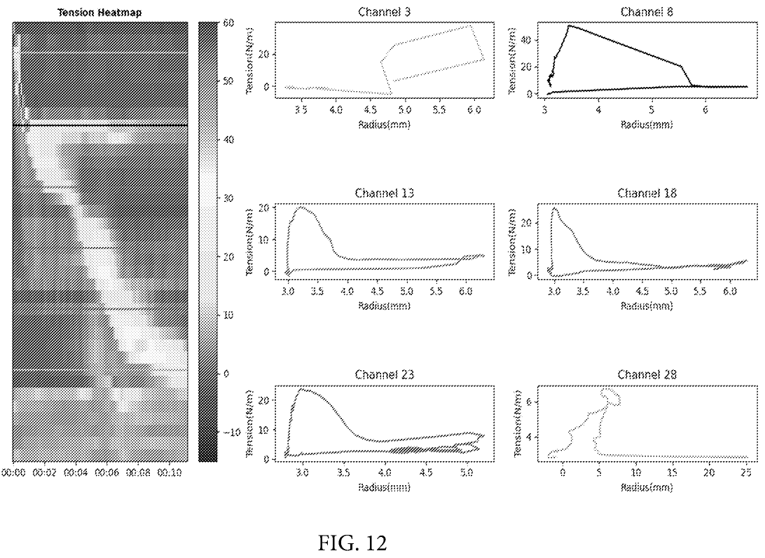

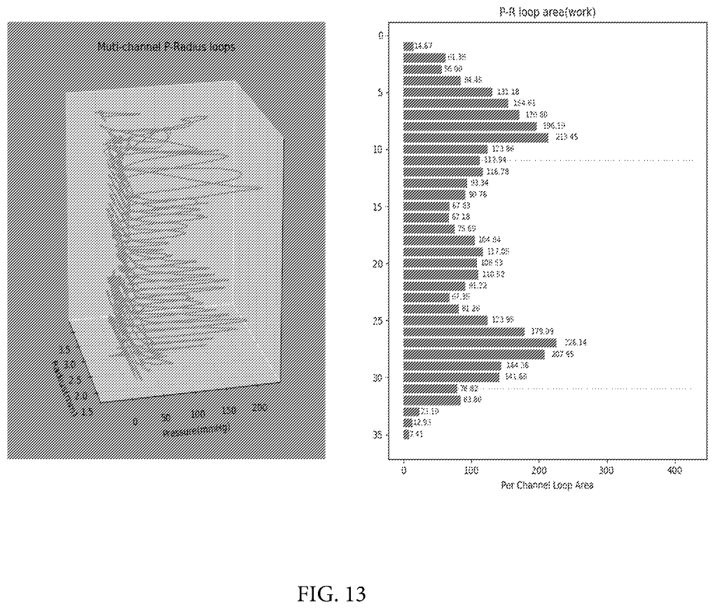

Additional features are shown in FIGS. 12 and 13. In particular, FIG. 12 shows illustrative esophageal length tension loops as previously described and FIG. 13 shows illustrative esophageal pressure-radius lofts as previously described.

The luminal CSA measurements obtained using the systems and methods described herein have been validated against a gold standard, i.e., intraluminal ultrasound images. Based on these systems and methods, the maximal luminal CSA anywhere in the esophagus has been determined to be approximately 200 mm². In contrast to these validated systems and methods, other techniques do not appear to have been equally validated. For instance, U.S. Pat. No. 10,143,416 uses a different algorithmic approach that requires the volume of the swallowed bolus in its calculation of the luminal CSA. In contrast, the algorithmic approaches described herein do not use the volume of the swallowed bolus as a parameter. Other techniques, such as described in WO 2012/034168 A1 for instance, use impedance and pressure measurements to assess oropharyngeal and esophageal motor functions. However, it does not show a technique for measuring the luminal cross-sectional area.

Various embodiments described herein may be described in the general context of method steps or processes, which may be implemented in one embodiment by a computer program product, embodied in, e.g., a non-transitory computer-readable memory, including computer-executable instructions, such as program code, executed by computers in networked environments. A computer-readable memory may include removable and non-removable storage devices including, but not limited to, Read Only Memory (ROM), Random Access Memory (RAM), compact discs (CDs), digital versatile discs (DVD), etc. Generally, program modules may include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

A computer program product can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network.

The various embodiments described herein may be implemented in various environments. Such environments and related applications may be specially constructed for performing the various processes and operations according to the disclosed embodiments or they may include a general-purpose computer or computing platform selectively activated or reconfigured by code to provide the necessary functionality. The processes disclosed herein are not inherently related to any particular computer, network, architecture, environment, or other apparatus, and may be implemented by a suitable combination of hardware, software, and/or firmware. For example, various general-purpose machines may be used with programs written in accordance with teachings of the disclosed embodiments, or it may be more convenient to construct a specialized apparatus or system to perform the required methods and techniques. In some cases the environments in which various embodiments described herein are implemented may employ machine-learning and/or artificial intelligence techniques to perform the required methods and techniques.

The above examples and disclosure are intended to be illustrative and not exhaustive. These examples and description will suggest many variations and alternatives to one of ordinary skill in this art. For instance, while the examples described above has illustrated the systems and techniques described herein as being applicable to measurements associated with the esophagus, more generally these systems and techniques are equally applicable to any portion of the gastrointestinal tract. All these alternatives and variations are intended to be included within the scope of the attached claims. Those familiar with the art may recognize other equivalents to the specific embodiments described herein which equivalents are also intended to be encompassed by the claims attached hereto.

1. A method for determining one or more parameters associated with an esophagus, comprising:
receiving data measured by an impedance and high-resolution manometry catheter in an esophagus, the data representative of (i) an impedance or voltage associated with at least one swallowing event during which an amount of a bolus is consumed and (ii) a baseline impedance obtained in an absence of a bolus being consumed;
determining, based on the received data and conductivity value of the bolus, a cross-sectional area of the esophagus; and
revising a value of the cross-sectional area that is determined using a correction factor that is obtained in vitro by repeating the determining step to determine a cross-sectional area of tubes of known diameter.

2. The method of claim 1, wherein receiving the data recorded by the impedance and high-resolution manometry catheter includes receiving data representative of an impedance associated with a first swallowing event during which a known amount of a first bolus is consumed, and an impedance associated with a second swallowing event during which a known amount of a second bolus is consumed, the first and second boluses having first and second conductivity values that are different from one another; and
determining the cross-sectional area of the esophagus based on the received data and the first and second conductivity values.

3. The method of claim 1, wherein the determining accounts for conductance of perimeter tissues and organs surrounding the esophagus.

4. The method of claim 1, wherein the determining assumes a conductance of zero for perimeter tissues and organs surrounding the esophagus.

5. The method of claim 1, wherein the recording is performed while a subject whose esophagus is being analyzed is lying down in the Trendelenburg position.

6. The method of claim 2, wherein the recording is performed while a subject whose esophagus is being analyzed is lying down in the Trendelenburg position.

7. The method of claim 1, wherein recording the data measured by the impedance and high-resolution manometry catheter includes recording data representative of pressure associated with the swallowing event.

8. The method of claim 7, further comprising determining values of tension in walls of the esophagus based on the cross-sectional area of the esophagus and the pressure.

9. The method of claim 7, further comprising determining values of distensibility in walls of the esophagus based on the cross-sectional area of the esophagus and the pressure.

10. The method of claim 8, further comprising generating a display of the cross-sectional area of the esophagus and the tension at a plurality of locations along a length of the esophagus.

11. The method of claim 7, further comprising generating a display that simultaneously includes a heatmap of the pressure and the impedance along a length of the esophagus.

12. The method of claim 7, further comprising generating a display that simultaneously includes impedance gradient streamlines overlayed on a heatmap of the pressure along a length of the esophagus.

13. The method of claim 7, further comprising generating a display that simultaneously includes esophageal distension and contraction during peristalsis at a plurality of points along a length of the esophagus and a plurality of different times.

14. The method of claim 13, wherein the distension and contraction are displayed as waveforms.

15. The method of claim 13, wherein distension is displayed as a waveform and pressure as heatmap.

16. The method of claim 13, wherein distension is displayed as a heatmap and pressure as a waveform.

17. The method of claim 7, further comprising generating a display that includes a cylindrical representation of distension and pressure at a plurality of points along a length of the esophagus at a plurality of different times during an entire swallow cycle.

18. The method of claim 9, comprising of generating a display that includes a cylindrical representation of distension and distensibility at a plurality of points along a length of the esophagus at a plurality of different times during an entire swallow cycle.

19. The method of claim 8, further comprising generating a display that includes a cylindrical representation of the distension and tension at a plurality of points along a length of the esophagus at a plurality of different times during an entire swallow cycle.

20. The method of claim 1, wherein the determining employs esophageal impedance tomography.

21. The method of claim 20, wherein the bolus is a liquid or solid bolus.

22. The method of claim 1, further comprising importing, visualizing, and analyzing esophageal or Gastrointestinal extracted parameters on a handheld or wearable device.

23. A non-transitory computer-readable medium, comprising instructions for causing a computing environment to perform a method for determining one or more parameters associated with a portion of the gastrointestinal tract, comprising:
receiving data measured by an impedance and high-resolution manometry catheter in a portion of a gastrointestinal tract, the data representative of (i) an impedance associated with at least one swallowing event during which an amount of a bolus is consumed and (ii) a baseline impedance obtained in an absence of a bolus being consumed, wherein receiving the data measured by the impedance and high-resolution manometry catheter includes receiving data representative of an impedance associated with a first swallowing event during which an amount of a first bolus is consumed and an impedance associated with a second swallowing event during which an amount of a second bolus is consumed, the first and second boluses having first and second conductivity values that are different from one another; and
determining, based on the received data and the first and second conductivity values of the bolus, a cross-sectional area of the portion of a gastrointestinal tract.
